(19) United States
(12) Patent Application Publication
(10) Pub. No.: US 2023/0372866 A1
(43) Pub. Date: Nov. 23, 2023

(54) CO2 Sequestration and Heavy Metal Fixing via Microbiological Precipitation of Calcium Carbonates

(71) Applicant: Douglas Bryant Rawson, Klamath Falls, OR (US)

(72) Inventor: Douglas Bryant Rawson, Klamath Falls, OR (US)

(21) Appl. No.: 17/664,423

(22) Filed: May 23, 2022

(51) Int. Cl. B01D 53/62; C01F 11/18; C01F 11/46; B01D 53/84

(52) U.S. Cl. CPC B01D 53/62; C01F 11/182; C01F 11/464; B01D 53/84; C01P 2006/37

(57) ABSTRACT

A method for sequestering CO₂ by creating precipitated calcium carbonates including Calcite, Dolomite, Vaterite and Struvite; (1) Utilizing a mutually beneficial bacterial/algal colony that can fix CO₂ as Calcite, Dolomite, Vaterite and Struvite (2) providing sunlight, water, CO₂ from either the air or industrial waste streams; and (3) assisting microbial/algal induced carbonate precipitation of Calcite, Dolomite, Vaterite and Struvite, thereby sequestering most of the CO₂ introduced in step (2). In addition, chlorine, sulfur, H₂S, NOx and toxic heavy metals will be fixed into the Calcite, Dolomite, Vaterite and/or Struvite matrix, rendering them environmentally harmless.

### BACKGROUND/SUMMARY

### Field of the Invention

The present invention relates to CO₂ sequestration and heavy metal fixing via the creation of Calcite, Dolomite, Vaterite and Struvite through bacterial/algal induced carbonate precipitation.

### Background of the Invention

The Intergovernmental Panel on Climate Change (IPCC) has presented compelling evidence that the accumulation of greenhouse gases (CO₂, CH₄ and N₂O) in the atmosphere has led to an increase in global temperatures over land of 1.5° C. This warming has contributed to the retreat of glaciers, sea-ice loss, and a mean sea level rise observed since the 1970s. It can be argued that climate change represents a tangible threat to our society.

There are innumerable sources of global CO₂ emissions; however, man-made greenhouse gas emissions have increased to due to recent economic and population growth. The atmospheric concentrations of carbon dioxide are at their highest levels in the last 500,000 years. As a result, immediate action is needed to stop and/or reverse the negative impacts of climate change on ecosystems and through them, on society. As a byproduct of fossil fuel combustion, chlorine gas, H₂S, NOx gas, sulfur, SO₂ and heavy metals can be found in flue streams of fossil fuel power plants.

Between 1780 and 2013, the cumulative anthropogenic CO₂ emissions were 2200 Gigatons of CO₂, 40% of which have remained in the atmosphere (900 Gigatons CO₂). While mitigating climate change can be done through policy changes which reduce annual global emissions, it will not reduce the current excess CO₂ in the atmosphere. To remove this excess CO₂ fixation and sequestration is required.

Current CO₂ fixation and sequestration (carbon capture and sequestration; CCS) uses three primary methods: 1) sequestration via biomass through photosynthesis; 2) sequestration as stable mineral carbonates generated using high pressure and high purity CO₂ gases; and 3) direct burial. There are benefits to using photosynthesis, including simplicity (planting trees, grasses, shrubs, algae), and converting biomass into biofuels or other industrially useful organic compounds There are drawbacks to the scale and complexity of current technologies. CCS of stable minerals requires high quality (99% pure or better CO₂), high temperatures (60° C. minimum.), and high pressures (150 bar minimum), where the CO₂ is injected into subsurface or waste alkaline minerals to produce carbonates. Burial is preferred due to simplicity and cost, although there are drawbacks, including the potential for CO₂ escape and inefficient CO₂ conversion to stable minerals.

Microbial/algal metabolic activity can promote biomineralization. Microbial/algal induced mineralization of Calcium Carbonate (CaCO₃) is one of the most studied mechanisms of biomineralization. The production of CaCO₃ minerals can allow for long-term storage of CO₂, as indicated by the 40 million Gigatons of CO₂ currently sequestered in carbonate rocks from the Carboniferous period. Historically studied metabolic activities that have been shown to drive microbial/algal CaCO₃ production include photosynthesis, urease, anhydrase, protease, denitrification, ammonification, sulfate reduction, and methane oxidation. Photosynthesis represents the most dominant natural mechanism for calcite biomineralization in the environment.

The present invention provides new methods for capture and sequestration of CO₂ and creation of precipitated calcium carbonates through microbial/algal induced carbonate precipitation. Additionally, toxic heavy metals created as by-products of fossil fuel combustion, mining and industrial processes are difficult to remove and can cause cancer and other deleterious effects on the human body, wildlife and the surrounding environment and an efficient method to remove and sequester these heavy metals is needed. This invention promotes immobilization, fixation and sequestration of chlorine, NOx, sulfur, H₂S, SO₂ and toxic heavy metals as a collateral effect makes this process ideal for flue-gas streams as well as being able to utilize contaminated water as the medium.

### SUMMARY OF THE INVENTION

An embodiment of the present invention provides a method for sequestering CO₂ and fixing heavy metals via the creation of precipitated Calcite, Dolomite, Vaterite or Struvite, the method comprising the steps of: (a) providing a liquid medium including: water, a carbon dioxide source (air injection or flue-stream injection), heavy metals and other contaminants and a symbiotic blend of bacteria/algae; (b) introducing sunlight to the liquid medium; and (c) allowing microbial/algal induced carbonate precipitation of calcium carbonate, dolomite, vaterite and struvite, thereby sequestering most of the CO₂, and nearly all the heavy metals introduced in said step of introducing.

Another embodiment of the present invention provides a method for sequestering CO₂ and creating precipitated Calcite, Dolomite, Vaterite or Struvite as in any embodiment above, wherein the liquid medium includes sugars, starches, amino acids, proteins, sulfur, SO₂, H₂S, NOx, chlorine, heavy metals, oxygen and sunlight.

Another embodiment of the present invention provides a method for sequestering CO₂ and creating precipitated Calcite, Dolomite, Vaterite or Struvite as in any embodiment above, wherein the liquid medium includes urea, ammonia, or urea and/or ammonia containing salts and/or calcium chloride, nitrates, nitrites or halide salts.

Another embodiment of the present invention provides a method for sequestering CO₂ and creating precipitated Calcite, Dolomite, Vaterite or Struvite as in any embodiment above, wherein the liquid medium is contained in a shallow pond (2-4 feet nominal depth).

Another embodiment of the present invention provides a method for sequestering CO₂ and creating precipitated Calcite, Dolomite, Vaterite or Struvite as in any embodiment above, wherein the liquid medium is contained in a reaction vessel consisting of a fibrous substrate and allowing the liquid medium to pass through said substrate capturing precipitated Calcite, Dolomite Vaterite or Struvite.

Another embodiment of the present invention provides a method for sequestering CO₂ and creating precipitated Calcite, Dolomite, Vaterite or Struvite as in any embodiment above, wherein the liquid medium is contained in a shallow pond with a mechanical apparatus such as a submerged conveyer or similar device used to collect the said precipitates. In addition, skimmers can be used to collect precipitated minerals directly from algal mats floating on the surface.

Another embodiment of the present invention provides a method for sequestering CO₂ and creating precipitated Calcite, Dolomite, Vaterite or Struvite as in any embodiment above, wherein the liquid medium includes carbohydrate sources, amino acid sources such as proteins and including acidifying carbohydrate sources that might lead to mixed acid fermentation, metabolic overflow and/or acetogenesis.

Another embodiment of the present invention provides a method for sequestering CO₂ and creating precipitated Calcite, Dolomite, Vaterite or Struvite as in any embodiment above, wherein the liquid medium includes waste-water effluent, cooling water effluent (either warm or cold) or other waste-water sources containing suspended solids including toxic metals, H₂S, sulfur, chlorine, sulphates, Helminths, Enterovirus or Enterobacteria.

Another embodiment of the present invention provides a method for sequestering CO₂ and creating precipitated Calcite, Dolomite, Vaterite or Struvite as in any embodiment above, wherein the bacteria are selected from the genera; Virgibacillus, Sporosarcina, Stenotrophomonas, Aspergillus, Myxococcus, Dehalobacter, Heliobacillus, Heliorestis, Ammonifex, Desulfovirgula, Halobacillus, or Chloracidobacterium.

Another embodiment of the present invention provides a method for sequestering CO₂ and creating precipitated Calcite, Dolomite, Vaterite or Struvite as in any embodiment above, wherein the bacteria are selected from the genera; Chromohalobacter, Halomonas, Marinobacter, Desulfovibrio, Pyrococcus, Pyrodictium, Salinivibrio, Sulfolobus, Desulfothermobacter, Thermoanaerobacter, Sulfobacillus, Sulfurimonas, Desulfurella.

Another embodiment of the present invention provides a method for sequestering CO₂ and creating precipitated Calcite, Dolomite, Vaterite or Struvite as in any embodiment above, wherein said step of providing a liquid medium comprises the steps of adjusting the pH of from pH 3 or more to pH 11 or less.

Another embodiment of the present invention provides a method for sequestering CO₂ and creating precipitated Calcite, Dolomite, Vaterite or Struvite as in any embodiment above, wherein any of the bacteria from the genera in any embodiment above are phenotypically or genotypically mutated to enhance or allow aerobic, photosynthetic, or metabolically increased precipitation efficiency.

Another embodiment of the present invention provides a method for sequestering CO₂ and creating precipitated Calcite, Dolomite, Vaterite or Struvite as in any embodiment above, wherein any of the bacteria from the genera in any embodiment above are combined with algae from the genera; Halimeda, Myxophyceae, Schizothrix, Scytonema, Microcoleus, Spirulina, Oscillatoria, Stigeoclonium, Cosmarium, Ulva, Halomicronema, Nannochloris, Porphyra, Emiliania.

Another embodiment of the present invention provides a method for sequestering CO₂ and creating precipitated Calcite, Dolomite, Vaterite or Struvite as in any embodiment above, wherein medium temperatures are controlled from 5° C. or more to 45° C. or less.

Another embodiment of the present invention provides a method for sequestering CO₂ and creating precipitated Calcite, Dolomite, Vaterite or Struvite as in any embodiment above, wherein medium containing bacteria/algae is exposed to natural or artificial sunlight for 2-10 hours per day.

Another embodiment of the present invention provides a method for sequestering CO₂ and creating precipitated Calcite, Dolomite, Vaterite or Struvite as in any embodiment above, wherein medium containing bacteria/algae is mechanically cleaned using filter medium or submerged conveyer to collect the precipitated Calcite, Dolomite, Vaterite or Struvite.

### DESCRIPTION

### Detailed Description of Illustrative Embodiments

The present invention provides a method for sequestering CO₂ and creating precipitated Calcite, Dolomite, Vaterite or Struvite, promoting calcification through a mutually beneficial symbiotic relationship between numerous bacteria and algae. Many of the microbes have been previously described in various works. Functionally this approach is different in that it utilizes phenotypically mutated bacteria in symbiosis with each other and along with algal genera to improve both calcium carbonate precipitation as well as heavy metal, sulfur, chlorine, nitrous oxides and hydrogen sulfide sequestration within the Calcite, Dolomite, Vaterite and Struvite matrix. Precipitated calcium carbonates (PCC) are recoverable from the liquid medium used in the CO₂ sequestration process and can have a number of industrial uses when processed properly.

In some embodiments, the present invention provides a method for sequestering CO₂ and creating precipitated calcium carbonates utilizing phenotypically mutated bacteria from the afore mentioned genera that are selected to metabolize Carbon Dioxide while using photosynthesis and aerobic respiration as opposed to the normal anaerobic metabolism found in wild strains. This leads to a five to ten (5-10) fold increase in efficiency and allows large surface-area, shallow ponds to be utilized.

In some embodiments, the present invention provides a method for sequestering CO₂ and creating precipitated calcium carbonates utilizing municipal waste-water effluent, industrial waste-water effluent, cooling water or other sources of water that does not meet federal, state or local purity standards for drinking water or direct discharge to lakes, rivers or streams.

In some embodiments, the present invention provides a method for sequestering CO₂ and creating precipitated calcium carbonates utilizing waste-water as mentioned above with flue-gas streams directly injected into the water, thus providing both aeration to assist aerobic bacterial action and a steady supply of CO₂ and other by-products such as H₂S, Sulfur, Chlorine, NOx and heavy metals.

In some embodiments, the present invention provides a method for sequestering CO₂ and creating precipitated calcium carbonates utilizing waste-water or other water sources as mentioned above with air directly injected into the water, thus providing both aeration to assist aerobic bacterial action and a steady supply of CO₂.

It has been demonstrated that the occurrence of bacterial alkalinization in protein rich media is due to spontaneous extracellular protein, or amino acid de-amination. When cells oxidize the carbon sources, the resulting product is comprised of CO₂ and water. Under alkaline conditions, CO₂ spontaneously evolves into CO₃. In addition, some bacterial species can accelerate the hydration of CO₂ through carbonic anhydrase activity. Additionally, calcium uptake and extrusion in bacteria are passive processes promoted by osmotic forces that maintain low intracellular calcium concentrations and, creates a highly saturated microenvironment near the cell wall and EPS. Bacteria produce CaCO₃ minerals where extra-polymeric substance (EPS), membrane, and cell wall structures act as nucleation sites. It appears, the metabolism of fatty acids plays a key role in biomineralization processes as well.

Previous work has examined Calcium Carbonate, Struvite, Vaterite and Dolomite precipitation by bacteria and algae and has shown that bacterial and algal cell surfaces show favorable conditions for calcium carbonite precipitation provided by the cell wall and extra polymeric substance. The mechanism involves carboxyl, phosphate, sulfate and hydroxyl functional groups of the cell walls and EPS become polarized or deprotonated and become negatively charged. They can then bind and accumulate cations of Ca₂⁺, Mg₂⁺, Cu²⁺, Fe³⁺, K⁺, Na⁺, Mn²⁺, Zn²⁺, Ni²⁺, As²⁺, Cd²⁺, Pb²⁺, Hg²⁺, Co²⁺ and others. When Ca²⁺ and Mg²⁺ levels reach high concentrations and local pH increases, CaCO₃ (Calcite and Vaterite) precipitates. The mechanism is the same for CaMg(CO₃)₂, (Dolomite) and MgNH₄PO₄·6H₂O (Struvite). This binding also allows heavy metal cations for example, but not limited to; Co²⁺, Ni²⁺, As²⁺, Cd²⁺, Pb²⁺, Hg²⁺ to bind with CO₃, thus becoming sequestered in the mineral matrix as NiCO₃ (Nickel Carbonate), AsCO₃ (Arsenic Carbonate), PbCO₃ (Lead Carbonate) etc. In addition, biomineralization can sequester toxic heavy metals directly into the mineral matrix by concretion of Calcium Carbonate around particles.

Previous research has shown that bacterial growth in the presence of glucose leads to the production of carboxylic and other organic acids, produced through a combination of glucose metabolic overflow, acetogenesis, and mixed acid fermentation which leads to the acidification of the media, and reduces the pH, preventing Calcium Carbonate precipitation. The present invention eliminates this problem by utilizing halophilic bacteria that reduce and metabolize carboxylic and other acids, thereby maintaining a higher pH near the Calcium Carbonate producing bacteria and algae. These halophilic bacteria such as Dehalobacter also sequester Chlorine and other Halogens, Halides and their organic salts.

Thus, in some embodiments, the liquid medium contains acidifying carbohydrate sources that lead to glucose metabolic overflow, acetogenesis, and mixed acid fermentation creating organic acids but not an increase in acidification.

In some embodiments, the bacteria are selected from one or more of the genera Virgibacillus, Sporosarcina, Stenotrophomonas, Aspergillus, Myxococcus, Dehalobacter, Heliobacillus, Heliorestis, Ammonifex, Desulfovirgula, Halobacillus, or Chloracidobacterium. In some embodiments, the bacteria are Virgibacillus. In some embodiments, bacteria are Sporosarcina. In some embodiments, the bacteria are Stenotrophomonas. In some embodiments the bacteria are Aspergillius. In some embodiments the bacteria are Myoxococcus. In some embodiments the bacteria are Dehalobacter. In some embodiments the bacteria are Heliobacillus. In some embodiments, the bacteria are Heliorestis. In some embodiments, the bacteria are Ammonifex (Nitrosomonas aestuarii-like ammonia oxidizing bacterium). In some embodiments the bacteria are Desulfovirgula. In some embodiments, the bacteria are Halobacillus. In some embodiments the bacteria are Chloracidobacterium, and in yet other embodiments, a combination of one or more or all of the above genera.

In some embodiments, the bacteria are selected from one or more of the genera Chromohalobacter, Halomonas, Marinobacter, Desulfovibrio, Pyrococcus, Pyrodictium, Salinivibrio, Sulfolobus, Desulfothermobacter, Thermoanaerobacter, Sulfobacillus, Sulfurimonas, Desulfurella. In some embodiments, the bacteria are Chromohalobacter. In some embodiments, bacteria are Halomonas. In some embodiments, the bacteria are Marinobacter. In some embodiments the bacteria are Desulfovibrio. In some embodiments the bacteria are Pyrococcus. In some embodiments the bacteria are Pyrodictium. In some embodiments, the bacteria are Salinivibrio. In some embodiments, the bacteria are Sulfolobus. In some embodiments the bacteria are Desulfothermobacter. In some embodiments the bacteria are Thermoanaerobacter. In some embodiments, the bacteria are Sulfobacillus. In some embodiments the bacteria are Sulfurimonas. In some embodiments the bacteria are Desulfurella and in yet other embodiments, a combination of one or more or all of the above genera.

In some embodiments, the algae are selected from one or more of the genera genera; Halimeda, Myxophyceae, Schizothrix, Scytonema, Microcoleus, Spirulina, Oscillatoria, Stigeoclonium, Cosmarium, Ulva, Halomicronema, Nannochloris, Porphyra, Emiliania. In some embodiments, the algae are Halimede. In some embodiments, algae are Myxophycea. In some embodiments, the algae are Schizothrix. In some embodiments the algae are Scytonema. In some embodiments the algae are Microcoleus. In some embodiments the algae are Spirulina. In some embodiments, the algae are Oscillatoria. In some embodiments the algae are Stigeoclonium. In some embodiments the algae are Cosmarium. In some embodiments, the algae are Ulva. In some embodiments the algae are Halomicronema. In some embodiments the algae are Nannochloris. In some embodiments the algae are Porphyra. In some embodiments the algae are Emiliania, and in yet other embodiments, a combination of one or more or all of the above genera.

In order to initiate colony start-up, a mixture of the bacterial species is inoculated into a culture of standard nutrient broth that contains 5.0-10.0 g/L of a pancreatic digest of gelatin and 4.0-6.0 g/L beef extract, and 15-25 g/L glucose. This broth is incubated at 18-42° C. with agitation at 180 rpm for 12-36 hours. This starter culture is then introduced into the liquid medium at 1:500-3,000 dilution. After introduction, once bacterial concentrations have reached minimum concentrations, (depending on the liquid medium characteristics) the algal spores can be introduced into the liquid medium. Algal spores can be directly added to the medium or pre-diluted in water. In some embodiments, CaCl₂) is added to the liquid medium if needed to maintain sufficient calcium and chlorine ions.

In some embodiments, to expedite the induced precipitation of calcium carbonate, the liquid medium is maintained at a suitable temperature for growth of the bacteria. In some embodiments, the liquid calcification medium is maintained with suitable aeration for growth of the bacteria. In some embodiments, the temperature of the liquid medium is maintained between 5° C. and 45° C.

In some embodiments the PCCs are separated from the liquid media via filtration, including membrane filtration or tangential flow filtration. In some embodiments, the PCCs are removed directly from the shallow pond via a conveyer system or similar extraction method. In some embodiments, the shallow ponds are allowed to evaporate and the PCCs are harvested before refilling and re-using the pond.

It has been demonstrated that the calcium salt chemistry and trace element presence both influence the type of Calcium Carbonate polymorph formed. Calcium acetate, lactate, and propionate, form a meta-stable polymorph vaterite. When Calcium succinate dominates, a combination of vaterite and calcite is formed, while using pyruvate, calcite is the dominant mineral. When magnesium is present, Dolomite can form, when magnesium and phosphates are present, Struvite forms. The metabolism of the carboxylic acids will influence the metabolic products produced, which in turn attach to the surface of the mineral and influence the type of polymorph precipitated. This allows tailoring of the final precipitate.

CO₂ can be sequestered as PCCs in the various shapes, compositions and sizes. calcium carbonate has a number of industrial applications: Carbonates serve as fillers, coatings, and modify the smoothness, brightness of paper products. Carbonates are used as filler to reduce polymer volume, modulate elasticity and increase impact resistance of plastics. Carbonates serves as filler, thixotropic agents, and reduce shrinkage during polymer setting. Carbonates serve as extenders, and are important for opacity, brightness, sheen and durability of paints. Over 110 megatons of carbonate were produced globally in 20219 using extraction (mining) technologies. The current invention produces Calcium Carbonates at volumes suitable to replace around 15% of the current extraction technologies while sequestering over one (1) gigaton of CO₂ per annum.

The present invention significantly advances the art of Carbon Capture and Sequestration (CCS) in a number of ways. While some embodiments of the invention have been disclosed herein, it should be noted that the invention is not limited thereto or thereby in-as-much as variations of this invention will be appreciated by those of ordinary skill in the field.

### Examples

Previous research has concluded that microorganisms bring about calcium carbonate precipitation by increasing the pH of media containing excess Ca²⁺ and dissolved HCO₃. However, recent research has led to a deeper understanding of metabolic calcification mechanisms.

Through the use of Sporosarcina Pasteurii, it can be shown that EPS and the bacterial cell surface provide the correct environment and nucleation sites for efficient Calcium Carbonate precipitation.

Calcification by Sporosarcina Pasteurii: In order to vastly improve the calcification efficiency of bacteria, this invention utilizes phenotypic mutations of common bacterial genera to select bacteria that are UV resistant, ambient temperature acclimatized in some cases aerobic, and in some embodiments aerobic and photosynthetic. The methods described below are applicable to most bacterial genera and some algal strains.

Bacteria are known to hydrolyze urea by urease for the purposes of; increasing the ambient pH, utilizing urea as a nitrogen source, and using it as a source of energy Sporosarcina Pasteurii is known to produce a large amount of urease in aqueous and soil environments. Additional hydrolysis reactions commonly used are protease and anhydrase reactions. The solubility of calcite is a function of pH and is affected by the ionic strength found in the aqueous medium. When urea, carbonic anhydrase or other HCO₃⁻ (bicarbonate) ion sources and calcium chloride or other metal salts are added to a media that supports microbial growth, the bacterial cell surface in conjunction with a variety of ions will induce mineral deposition by providing nucleation sites. Normally Ca₂⁺ ions (or other ions as mentioned in paragraph 30) are not utilized for metabolic processes, but rather accumulate outside the cell in the EPS. In some mediums it is possible that individual microbes will produce ammonia as a result of enzymatic urea hydrolysis and create an alkaline micro-environment around the cell. The high pH of these localized areas, without an increase in pH of the entire medium will initiate the growth of calcite, dolomite or struvite crystals around the cell.

In addition to urease activity, a similar mechanism as the one described above occurs with protease as well. Some bacteria also utilize carbonic anhydrase. In addition, it is a fact that facultatively anaerobic S. pasteurii grows at a higher rate in the presence of oxygen and consequently induces active precipitation of CaCO₃ more effectively

The influence of extracellular polymeric substance (EPS) secretions on calcium carbonate precipitation is as follows: EPS plays an important role in the coverage of the surface by biofilms, cell adhesion and possibly the capturing of any produced calcium carbonate. This can result in a homogeneous layer of calcium carbonate. A biofilm can colonize the surface of the cells and act as nucleation site for extracellular calcium carbonate precipitation. The structure of the biofilm is influenced by a number of biological factors, such as twitching motility, growth rate, cell signaling, and EPS production. The biofilm structure appears to be largely determined by the production of a slime-like matrix of EPS, which provides the structural support for the biofilm.

Improvement of bacterial and algal strains for increased production of proteins or enzymes, amino acids or other desired compounds has been essential for most commercial bacterial/algal processes. Such improved strains can reduce the cost of the process while increasing productivity and may also possess specialized desirable characteristics. The physical mutagenesis by UV radiation in strain improvement for enhanced productivity and calcite precipitation is demonstrated in this invention. A significant increase in the urease activity, calcite amount, and survival rate at higher pH levels is observed in case of UV-induced mutants of S. pasteurii compared with the wild type. These results suggest that the mutant strain of S. pasteurii can be exploited commercially and that other similar strains as mentioned in the previous sections of this invention can be similarly mutated and improved. Different methods of phenotypical mutation were utilized for various types of bacteria. Bacteria that are thermophilic were mutated using colder temperatures in order to select strains that can survive below 37° C. Anaerobic bacteria were exposed to varying levels of oxygen and UV radiation until specific strains that can survive in air were isolated. Bacteria that are normally helio-phobic were exposed to sunlight and UV radiation in order to isolate strains that survive in sunlight.

Bacteria are often considered as isolated individuals, incapable of organized behaviors observed in multicellular organisms. However, as W. Hastings, discovered twenty years ago, some bacteria have the capacity to emit light and this ability is entirely determined by their relative number. These bacteria have to be concentrated enough to produce light. It appears as if they could measure their own numbers to induce expression of the genes necessary for light production. This is known in general, as a “quorum”, hence the name “quorum sensing” has been used to describe this phenomenon. According to W. Hastings and others, “quorum sensing is a mechanism of communication between bacteria that leads them to display an organized collective behavior”. This Quorum sensing also exists for other processes. When the concentration reaches a threshold value, the effects of “quorum sensing” begin to control a large number of processes, such as: luminescence, transfer of plasmids, collective movement of microbes or synthesis of some reserve molecules. By combining the afore mentioned bacteria and algal strains, not only does each microorganism contribute its main individual characteristic such as sulfur digesting, calcium precipitating, nitrogen fixing, photosynthesis, heavy metal sequestering, nitrate digestion, glucose production or other, the colony uses quorum sensing to improve the overall efficiency of these process, resulting in large-scale symbiosis.

### Methods

Initial Selection and Acquisition of Bacterial Strains

Sporosarcina pasteurii (Miguel) Yoon et al. (ATCC 11859) was obtained in pellet form from ATCC (Manassas Virginia, USA). Additional samples of the following bacteria were also obtained: Virgibacillus pantothenticus (Proom and Knight) Heyndrick et al. (ATCC 14576). Chromohalobacter marismortui Ventosa et al. (ATCC 17056). Stenotrophomonas maltophilia (Hugh) Palleroni and Bradbury (ATCC 700269). Myxococcus xanthus Beebe (ATCC 27922). Desulfovibrio desulfuricans subsp. desulfuricans (Beijerinck) Kluyver and van Niel (ATCC 27774). Pyrococcus sp. (ATCC BAA-2246). Pyrodictium delaneyi (ATCC BAA-2559. Halomonas halodenitrificans (Robinson and Gibbons) Dobson and Franzmann (ATCC 12084). Halomonas halophila (Quesada et al.) Dobson and Franzmann (ATCC 49969). Marinobacter hydrocarbonoclasticus Gauthier et al. (ATCC 49840). Marinomonas communis (Baumann et al.) van Landschoot and De Ley (ATCC 27118). Salinivibrio costicola subsp. costicola (Smith) Mellado et al. (ATCC 43148). Pseudomonas fluorescens Migula (ATCC 13525). Aspergillus nidulans (Eidam) Winter (ATCC 38163). Sulfolobus solfataricus Zillig et al. (ATCC 35092). Heliorestis convoluta (ATCC BAA-1281) Nitrosomonas aestuarii-like ammonia oxidizing bacterium (ATCC PTA-5423). Chloracidobacterium thermophilum (ATCC BAA-2647). Desulfothermobacter ferriducens (ATCC TSD-233). Thermoanaerobacter pseudethanolicus (ATCC 33223). Sulfobacillus acidophilus Norris et al. (ATCC 700253). Sulfurimonas denitrificans (ATCC 33889). Desulfurella acetivorans Bonch-Osmolovskaya et al. (ATCC 51451).

Initial Selection and Acquisition of Algal Strains.

The following algal strains were obtained from UTEX (Austin TX USA): UTEX B 1818 Schizothrix calcicola var. vermiformis. UTEX B 1834 Scytonema hofmanni. UTEX B 1815 Microcoleus vaginatus var. cyano-viridis. UTEX LB 2342 Spirulina maxima. UTEX B EE24 Oscillatoria sp. UTEX B 441 Stigeoclonium helveticum. UTEX LB 1043 Cosmarium turpinii. UTEX LB 1860 Ulva fasciata. UTEX B 3008 Halomicronema excentricum. UTEX 2502 Nannochloris eucaryotum. UTEX 161 Porphyridium cruentum. UTEX LB 1016 Emiliania huxleyi.

After initial re-animation, the bacteria were maintained at 37° C. in nutrient medium (pH 8.0). Bacteria that are normally anaerobic, thermophilic, helio-phobic or otherwise extreme environment adapted were cultured using the ATCC medium suggested in the ATCC literature to yield viable cultures before being phenotypically mutated using various methods afore mentioned. After mutagenesis was carried out for all strains, they were stored at 37° C. in nutrient medium before being utilized.

The phenotypical mutation of S. pasteurii was carried out via UV irradiation. S. pasteurii was grown for 12 hours in nutrient broth containing 2% urea solution at 37° C. under mild, constant shaking. The cells were washed twice with 0.5 Mole phosphate buffer solution (pH 8.0) and re-suspended in 10 ml of the same buffer. The cells were diluted in phosphate buffer to obtain about 5×10⁸ cfu/ml and exposed to UV light using a 20-watt Philips germicidal lamp for 30 min, where a less than 5% survival rate was observed. The surviving colonies were transferred onto urea agar base media to check the production of urease. These mutants were re-cultured seven times to assure that no reverse mutation had occurred before utilization of the mutated strains.

For the S. pasteurii verification, all chemicals and growth media were obtained from Fisher Scientific (Pittsburgh, Pa.). A starting liquid media (8 g nutrient broth, 10 g glucose, 2 g urea and 2.5 g CaCl₂) per liter) was heated to 37° C. and the mutated bacterial strains added.

Verification and Measurement of Calcification Rates Using Solid Media

As calcium carbonate precipitation occurs rapidly at 37° C., mutated S. pasteurii, was grown at slightly less than ambient temperature (18° C.) to assess calcium carbonate precipitation rates. Agar B4 was inoculated with the bacteria and after four days, twenty individual colonies were chosen at random and imaged using an optical microscope equipped with a camera. Images were captured using cellSens Standard software and analyzed using TWS software v3.2.34) and MorphoLibJ to measure calcification. The software was programmed to identify calcium carbonate, dolomite or struvite crystals using a set of baseline images of colonies from similar plates, followed by manually designating areas as crystals, colonies, or background agar to generate an overlay. The classification was then compared with other images of similar morphology, which was further refined with additional training images. Once precipitates had been identified, the percent coverage was calculated using Analyze Particles functions built into ImageJ software.

Precipitate Analysis

The analysis of carbonate minerals was carried out using cultures of mutated S. pasteurii grown as mentioned above and inoculated into 10 mL of liquid media in a 100 mL flask and grown for one week at room temperature with shaking at 100 rpm. All precipitated minerals were collected by vacuum filtration using Millipore 6.0 μm MCE membrane and washed with buffer at pH 8.0. The filters were allowed to dry for 2 hours at 25° C. in glass Petri plates. The surface of the membranes was then scraped with a metal spatula and allowed to dry for an additional 36 hours. This material was then subjected to XRD analysis to determine crystal lattice structures and chemical composition

1. A method for sequestering CO₂ by creating precipitated calcium carbonates including Calcite, Dolomite, Vaterite and Struvite: (1) utilizing a mutually beneficial bacterial/algal colony composed of bacteria that can fix CO₂ as Calcite, Dolomite, Vaterite and Struvite (2) providing sunlight, water, CO₂ from either the air or industrial waste streams; and (3) assisting microbial/algal induced carbonate precipitation of Calcite, Dolomite, Vaterite and Struvite, thereby sequestering most of the CO₂ introduced in step (2).

2. A method wherein, chlorine, sulfur, H₂S and NOx form flue-gas streams, waste water or other sources will be digested/converted by a symbiotic bacterial/algal colony to produce sugars, starches, proteins, elemental solids, amino acids and other essential ions to help support colony health and growth.

3. A method wherein toxic heavy metals from waste water, flue-gas streams, or other sources will be fixed into the Calcite, Vaterite, Dolomite and/or Struvite matrix, rendering them environmentally harmless.

4. The method of claim 1-3, wherein the bacteria are selected from one or more of the genera Virgibacillus, Sporosarcina, Stenotrophomonas, Aspergillus, Myxococcus, Dehalobacter, Heliobacillus, Heliorestis, Ammonifex, Desulfovirgula, Halobacillus, Chloracidobacterium, Chromohalobacter, Halomonas, Marinobacter, Desulfovibrio, Pyrococcus, Pyrodictium, Salinivibrio, Sulfolobus, Desulfothermobacter, Thermoanaerobacter, Sulfobacillus, Sulfurimonas, or Desulfurella.

5. The method of claim 1-3, wherein the algae are selected from one or more of the genera; Halimeda, Myxophyceae, Schizothrix, Scytonema, Microcoleus, Spirulina, Oscillatoria, Stigeoclonium, Cosmarium, Ulva, Halomicronema, Nannochloris, or Porphyra, Emiliania.

6. The method of claim 1-3, wherein the liquid medium includes waste-water effluent, cooling water effluent (either warm or cold) or other waste-water sources containing suspended solids including heavy metals, H₂S, sulfur, chlorine, sulphates, Helminths, Enterovirus or Enterobacteria.

7. The method of claim 1-3, wherein phenotypically mutated bacteria are utilized from the afore mentioned genera and are selected to metabolize Carbon Dioxide while using photosynthesis and aerobic respiration.

8. The method of claim 1-3, wherein phenotypically mutated bacteria and or algae are utilized from the afore mentioned genera and are selected to metabolize chlorine, nitrates, nitrites, sulfur, phosphates, proteins, starches, urea, amino acids, glucose hydrogen sulfide, NOx using photosynthesis, aerobic respiration, metabolic digestion, glucose production or heavy element binding.

9. The method of claim 1-3, wherein the liquid medium utilizes waste-water or other water sources as mentioned above with air directly injected into the water, thus providing both aeration to assist aerobic bacterial action and a steady supply of CO₂.

10. The method of claim 1-3, wherein flue-gas streams are directly injected into the water, thus providing both aeration to assist aerobic bacterial action and a steady supply of CO₂ and other by-products such as H₂S, Sulfur, Chlorine, NOx and heavy metals.

11. The method of claim 1-3, wherein the liquid medium includes urea, ammonia, or urea and/or ammonia containing salts and/or calcium chloride, nitrates, nitrites, sulfides, halogens, proteins, glucose, phosphates, or halide salts.

12. The method of claim 1-3, wherein the liquid medium is contained in a shallow pond (2-4 feet nominal depth).

13. The method of claim 1-3, wherein the liquid medium is contained in a reaction vessel consisting of a fibrous substrate and allowing the liquid medium to pass through said substrate capturing precipitated Calcite, Vaterite, Dolomite or Struvite.

14. The method of claim 1-3, wherein a mechanical apparatus such as a submerged conveyer or similar device used to collect the said precipitates or skimmers are used to collect precipitated minerals directly from algal mats floating on the surface.

15. The method of claim 1-3, wherein the liquid medium includes carbohydrate sources, amino acid sources such as proteins and including acidifying carbohydrate sources that might lead to mixed acid fermentation, metabolic overflow and/or acetogenesis.

16. The method of claim 1-3, wherein the liquid medium comprises the steps of adjusting the pH of from pH 3 or more to pH 11 or less.

17. The method of claim 1-3, wherein the liquid medium is maintained with suitable aeration for growth of the bacteria.

18. The method of claim 1-3, wherein, the temperature of the liquid medium is maintained between 5° C. and 45° C.

19. The method of claim 1-3, wherein the shallow ponds are allowed to evaporate and the PCCs are harvested before refilling and re-using the pond.

20. The method of claim 1-3, wherein the liquid medium includes sugars, starches, sulfur, SO₂, H₂S, NOx, chlorine, heavy metals, oxygen and sunlight.
